(19) United States
(12) Patent Application Publication
(10) Pub. No.: US 2024/0343669 A1
(43) Pub. Date: Oct. 17, 2024

(54) APPLICATION OF METAL SULFIDE PIEZOELECTRIC MATERIAL IN PIEZOELECTRIC CATALYTIC REDUCTION OF CO2

(71) Applicant: SOOCHOW UNIVERSITY, Suzhou (CN)

(72) Inventors: Jianmei LU, Suzhou (CN); Najun LI, Suzhou (CN)

(21) Appl. No.: 18/294,273

(22) PCT Filed: Aug. 22, 2022
(86) PCT No.: PCT/CN2022/113990

§ 371 Date: Feb. 1, 2024

(30) Foreign Application Priority Data
Mar. 16, 2022 (CN) 202210262441.3

(51) Int. Cl. C07C 51/353; B01J 27/04; B01J 27/051; B01J 35/33; B01J 37/04; B01J 37/06; B01J 37/20

(52) U.S. Cl. CPC C07C 51/353; B01J 27/04; B01J 27/051; B01J 35/33; B01J 37/04; B01J 37/06; B01J 37/20

(57) ABSTRACT

Disclosed in the present invention is an application of a metal sulfide piezoelectric material in piezoelectric catalytic reduction of CO₂. The internal electric field generated by the piezoelectric material under external mechanical stimulation pushes electrons and holes to migrate in opposite directions, so that natural advantages are provided for separation and transmission of electron-hole pairs, but there is no report in the prior art that CO₂ is reduced into C2+ products by means of piezoelectric catalysis. The present invention provides a metal sulfide piezoelectric nanomaterial and a preparation method therefor. The metal sulfide piezoelectric nanomaterial is used for piezoelectric catalytic reduction of CO₂ for the first time, especially piezoelectric catalytic reduction of CO₂ to obtain C2+ products so as to broaden the technical route of catalytic reduction of CO₂.

### TECHNICAL FIELD

The present invention relates to the field of piezoelectric nanomaterials and piezoelectric catalysis technology, specifically to the preparation method of a kind of metal sulfide piezoelectric nanomaterial and the application in piezoelectric catalytic reduction of CO₂.

### BACKGROUND OF INVENTION

Due to the rapid growth of the world population and the rapid development of global industrialization, the demand for fossil fuels has sharply increased. The large amount of accompanying carbon dioxide emissions has led to adverse global climate change, and there is an urgent need to develop renewable and clean energy. At present, it is possible to use renewable solar or electric energy to drive the synthesis of CO₂ and H₂O to produce Cl products (CO, formic acid, and methane). However, it's still greatly challengeable to selectively generate the valued C2+products such as ethanol and acetic acid through direct reduction of CO₂ due to the difficulty of C—C coupling process and the necessity of dual active catalytic sites. In addition, most reported CO₂ reduction systems suffer from low conversion efficiency and uncontrollable selectivity. Therefore, there is an urgent need to develop new green economy catalytic conversion technologies to achieve efficient and highly selective catalytic systems for reducing CO₂ to C2+products.

### Technical Problems

The new technology of piezoelectric catalysis has got high attention for converting ubiquitous mechanical energy into chemical energy, and has been widely applied in the removal of organic pollutants, decomposition of aquatic hydrogen, and many other aspects. The internal electric field generated by piezoelectric materials under external mechanical stimulation drives electrons and holes to migrate in the opposite direction, providing a natural advantage for the separation and transmission of electron-hole pairs. However, the existing technologies have not provided guidance on piezoelectric catalysis for reducing CO₂ to C2+products. The purpose of the present invention is to provide a metal sulfide piezoelectric nanomaterial and its preparation method, and to apply it for the first time for piezoelectric catalytic reduction of CO₂, especially for piezoelectric catalytic reduction of CO₂ to produce C2+products, in order to broaden the existing technical route.

### Technical Solution

In order to achieve the above objective, the present invention adopts the following technical solution: the application of metal sulfide piezoelectric material in piezoelectric catalytic reduction of CO₂.

The application of metal sulfide piezoelectric material in piezoelectric catalytic reduction of CO₂ to prepare C2+products.

A method for piezoelectric catalytic reduction of CO₂ introduces carbon dioxide into a solution containing metal sulfide piezoelectric material, and completes the piezoelectric catalytic reduction of CO₂ under mechanical force.

A method for piezoelectric catalytic reduction of CO₂ to prepare C2+products introduces carbon dioxide into a solution containing metal sulfide piezoelectric material, and completes the piezoelectric catalytic reduction of CO₂ under mechanical force to prepare C2+products.

In the present invention, the metal sulfide piezoelectric materials include metal sulfides, modified metal sulfides, and doped metal sulfides. That is, the present invention uses metal sulfides as piezoelectric materials, or any modified or doped metal sulfides as piezoelectric materials, as catalytic materials for piezoelectric catalytic reduction of CO₂; preferably, among the metal sulfides, the metal is one or several of conventional metal, transition metal, or precious metal, and further preferably, the metal includes one or several of molybdenum, cadmium, tin, zinc, copper, iron, silver, selenium, cobalt, nickel, and tungsten. Doping can be rare earth metal doping, and modification can be organic modification, such as metal sulfide organic composite piezoelectric materials. Conventional metals refer to metals other than transition metals, precious metals, and rare earth metals.

In the present invention, metal sulfides are prepared from corresponding metal salts and sulfur sources. As an example, in order to obtain metal molybdenum sulfides, the molybdenum salts used include either or both of ammonium molybdate and sodium molybdate; In order to obtain metal cadmium sulfides, the cadmium salts used include one or several of cadmium nitrate, cadmium chloride, cadmium acetate, and cadmium sulfate; In order to obtain metal tin sulfides, the tin salts used include one or two of tin tetrachloride and tin dichloride; In order to obtain metal zinc sulfides, the zinc salts used include one or several of zinc chloride, zinc nitrate, zinc acetate, and zinc sulfate; The sulfur source is one or several of thiourea, thioacetamide, mercaptan, and L-cysteine.

In the present invention, metal sulfides are prepared from corresponding metal salts and sulfur sources by hydrothermal or solvothermal method.

In the present invention, a solution containing metal sulfide piezoelectric materials includes metal sulfide piezoelectric materials, sacrificial agents and solvents, and does not contain other catalysts.

During the piezoelectric catalytic reduction of CO₂ by the metal sulfide piezoelectric material of the present invention, the external mechanical force is applied in the dark conditions to stimulate the internal polarization of the piezoelectric catalyst to generate free charge carriers (active electrons), achieving the goal of generating C2+products through piezoelectric catalytic reduction of CO₂.

### Beneficial Effects

The advantages of the present invention:

(1) The metal sulfide piezoelectric nanomaterials are applied in the piezoelectric catalytic system of CO₂ for the first time. In the dark conditions, mechanical energy can be converted into chemical energy to excite and generate active electrons, and a built-in electric field is formed inside the material to achieve the piezoelectric catalytic conversion of CO₂.

(2) The present invention achieves the piezoelectric catalytic conversion from CO₂ to C2+products for the first time. Under the condition of only applying mechanical force to generate piezoelectric catalysis without the need for photocatalysis or electrocatalysis, the metal sulfide piezoelectric material disclosed in the present invention can efficiently reduce CO₂ to obtain the only C2+product of acetic acid.

(3) The present invention adopts a simple and feasible material preparation method, and the material performance is stable, reusable, and cost-effective.

(4) The efficient, economical, and environmentally friendly piezoelectric catalytic technology in the present invention provides a way for utilizing environmental mechanical energy to reduce CO₂ to C2+products.

### BRIEF DESCRIPTION OF THE DRAWINGS

FIG. 1 shows the scanning electron micrographs of molybdenum sulfide, tin disulfide, and tin monosulfide prepared in Examples 1, 3, and 4, (a) molybdenum sulfide, (b) tin disulfide, and (c) tin monosulfide.

FIG. 2 shows the X-ray electron spectra of molybdenum sulfide, tin disulfide, and tin monosulfide prepared in Examples 1, 3, and 4, (a-b) molybdenum sulfide, (c-d) tin disulfide, and (e-f) tin monosulfide.

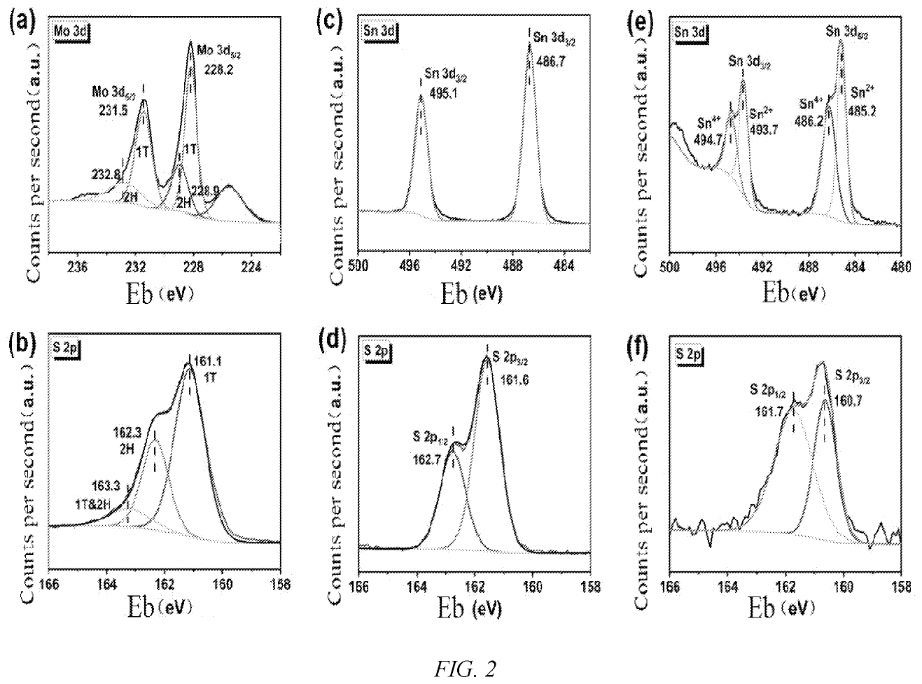

FIG. 3 shows the amplitude and phase curves of piezoelectric property of molybdenum sulfide, tin disulfide, and tin monosulfide prepared in Examples 1, 3, and 4, (a-b) molybdenum sulfide, (c-d) tin disulfide, and (e-f) tin monosulfide.

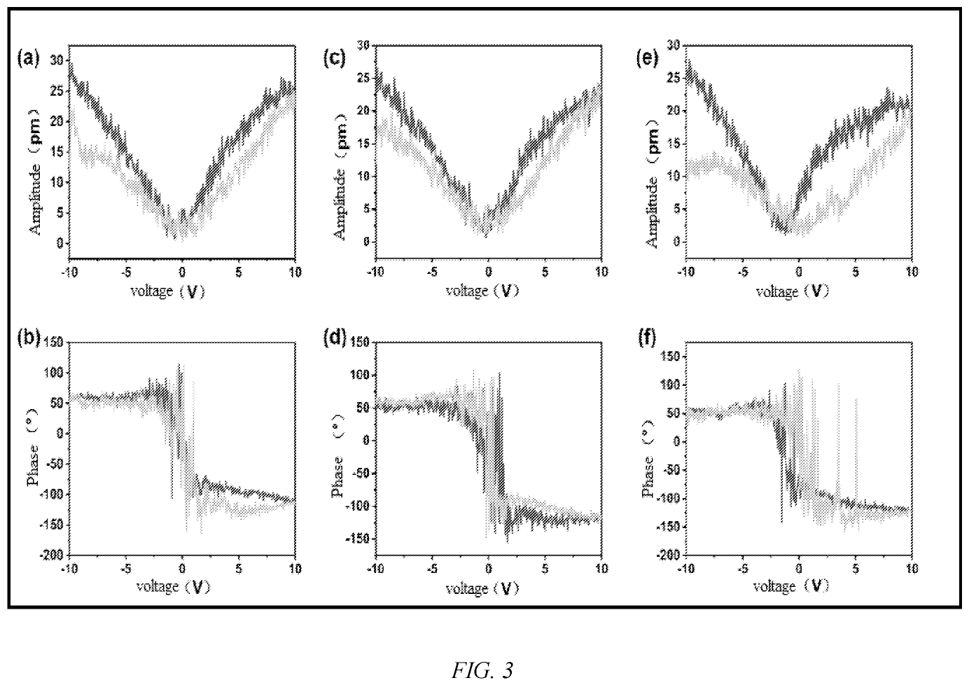

FIG. 4 shows the effect diagram of the piezoelectric catalytic reduction of CO₂ by molybdenum sulfide, tin disulfide, and tin monosulfide prepared in Examples 1, 3, and 4.

FIG. 5 shows the nuclear magnetic spectrum of product.

### EXAMPLES OF THE PRESENT INVENTION

The present invention uses a conventional and simple hydrothermal or solvothermal method to obtain a kind of metal sulfide piezoelectric nanomaterial, specifically including molybdenum sulfide, cadmium sulfide, tin sulfide, and zinc sulfide. In the dark conditions, only ultrasonic vibration is provided to achieve piezoelectric catalytic reduction of CO₂.

In the present invention, metal sulfides are prepared from corresponding metal salts and sulfur sources by hydrothermal or solvothermal method. Firstly, the precursors of corresponding metal ions (molybdenum, cadmium, tin, and zinc) are provided, and then a sulfur source is added to obtain a precursor solution and the solution is one of deionized water, anhydrous ethanol and ethylene glycol or the combination of the two. Correspondingly, the method using deionized water as the solvent is hydrothermal method; the method using anhydrous ethanol or ethylene glycol as the solvent is solvothermal method. Then, the high-temperature hydrothermal or solvothermal reaction of the precursor solution mentioned above is carried out. After the reaction is completed, the final product is obtained by washing with deionized water or anhydrous ethanol and drying.

As an example, in order to obtain metal molybdenum sulfide, a precursor solution with a molar mass ratio of molybdate and sulfur source at 1:1-40 was provided. Preferably, the deionized water was used as the solvent, ammonium molybdate was used as the molybdenum source, and thiourea was used as the sulfur source. The precursor solution was placed in a 50 mL PTFE lined reactor to react at 120-200° C. for 12-24 hours. After the reaction, the product was washed three times with deionized water and ethanol sequentially, and finally dried at 60° C. for 12 hours to obtain molybdenum sulfide. The obtained material was used for piezoelectric catalytic reduction of CO₂.

As an example, in order to obtain metal cadmium sulfide, a precursor solution with a molar mass ratio of cadmium salt and sulfur source at 1:1-2 was provided. Preferably, the deionized water was used as the solvent, cadmium acetate was used as the cadmium source, and thioacetamide was used as the sulfur source. The precursor solution was placed in a 50 mL PTFE lined reactor to react at 50-150° C. for 1-10 hours. After the reaction, the product was washed three times with deionized water and ethanol sequentially, and finally dried at 60° C. for 12 hours to obtain cadmium sulfide. The obtained material was used for piezoelectric catalytic reduction of CO₂.

As an example, in order to obtain metal tin sulfide, a precursor solution with a molar mass ratio of tin salt and sulfur source at 1:1-8 was provided. Preferably, the anhydrous ethanol and ethylene glycol were used as the solvent, tin tetrachloride and tin dichloride were used as the tin source, and thioacetamide was used as the sulfur source. The precursor solution was placed in a 50 mL PTFE lined reactor to react at 100-200° C. for 12-24 hours. After the reaction, the product was washed three times with deionized water and ethanol sequentially, and finally dried at 60° C. for 12 hours to obtain tin disulfide. The obtained material was used for piezoelectric catalytic reduction of CO₂.

As an example, in order to obtain metal zinc sulfide, a precursor solution with a molar mass ratio of zinc salt and sulfur source at 1:1-4 was provided. Preferably, the ethylene glycol was used as the solvent, zinc nitrate was used as the zinc source, and thioacetamide was used as the sulfur source. The precursor solution was placed in a 50 mL PTFE lined reactor to react at 100-200° C. for 12-24 hours. After the reaction, the product was washed three times with deionized water and ethanol sequentially, and finally dried at 60° C. for 12 hours to obtain zinc sulfide. The obtained material was used for piezoelectric catalytic reduction of CO₂.

The following is a further detailed description of the invention in conjunction with Examples and drawings, but the Examples of the present invention are not limited to these.

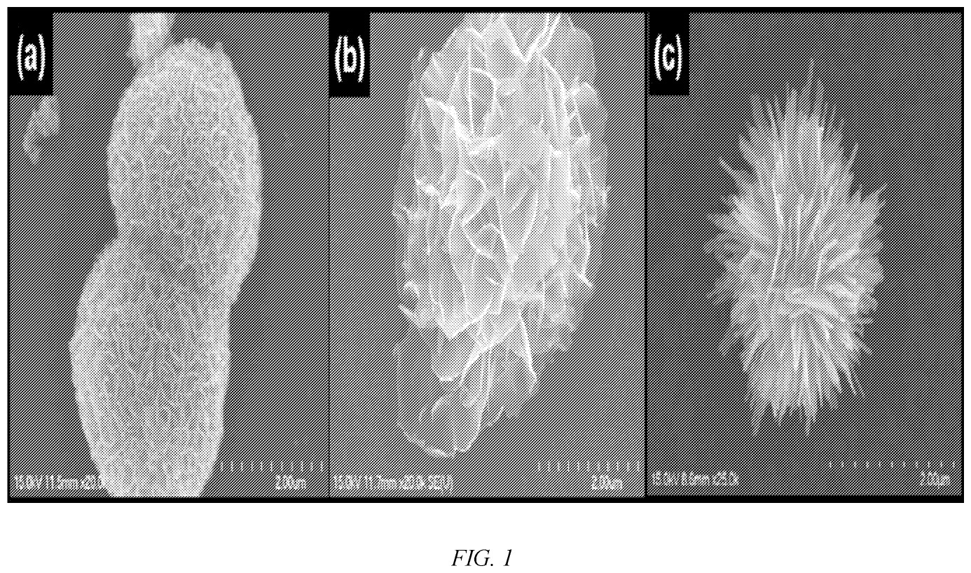

Example 1:40 mL of precursor solution with a molar mass ratio of molybdate salt and sulfur source at 1:35 was provided. The deionized water was used as the solvent, ammonium molybdate was used as the molybdenum source, and thiourea was used as the sulfur source. The precursor solution was placed in a 50 mL PTFE lined reactor to react at 200° C. for 24 hours. After the reaction, the product was washed three times with deionized water and ethanol sequentially, and finally dried at 60° C. for 12 hours to obtain molybdenum sulfide (see FIG. 1a). The obtained material was used for piezoelectric catalytic reduction of CO₂.

Example 2:40 mL of precursor solution with a molar mass ratio of cadmium salt and sulfur source at 1:1 was provided. The deionized water was used as the solvent, cadmium acetate was used as the cadmium source, and thioacetamide was used as the sulfur source. The precursor solution was placed in a 50 mL PTFE lined reactor to react at 90° C. for 2 hours. After the reaction, the product was washed three times with deionized water and ethanol sequentially, and finally dried at 60° C. for 12 hours to obtain cadmium sulfide. The obtained material was used for piezoelectric catalytic reduction of CO₂.

Example 3:40 mL of precursor solution with a molar mass ratio of tetravalent tin salt and sulfur source at 1:4 was provided. The anhydrous ethanol was used as the solvent, tin tetrachloride was used as the tin source, and thioacetamide was used as the sulfur source. The precursor solution was placed in a 50 mL PTFE lined reactor to react at 160° C. for 12 hours. After the reaction, the product was washed three times with deionized water and ethanol sequentially, and finally dried at 60° C. for 12 hours to obtain tin disulfide (see FIG. 1b). The obtained material was used for piezoelectric catalytic reduction of CO₂.

Example 4:40 mL of precursor solution with a molar mass ratio of bivalent tin salt and sulfur source at 1:1 was provided. The ethylene glycol was used as the solvent, tin dichloride was used as the tin source, and thioacetamide was used as the sulfur source. The precursor solution was placed in a 50 mL PTFE lined reactor to react at 120° C. for 24 hours. After the reaction, the product was washed three times with deionized water and ethanol sequentially, and finally dried at 60° C. for 12 hours to obtain tin monosulfide (see FIG. 1c). The obtained material was used for piezoelectric catalytic reduction of CO₂.

Example 5: The precursor solution with a molar mass ratio of zinc salt and sulfur source at 1:2 was provided. The ethylene glycol was used as the solvent, zinc nitrate was used as the zinc source, and thioacetamide was used as the sulfur source. The precursor solution was placed in a 50 mL PTFE lined reactor to react at 150° C. for 24 hours. After the reaction, the product was washed three times with deionized water and ethanol sequentially, and finally dried at 60° C. for 12 hours to obtain zinc sulfide. The obtained material was used for piezoelectric catalytic reduction of CO₂.

Example 6:10 mg of metal sulfide was taken and dispersed in 10 mL of sodium sulfite (Na₂SO₃) aqueous solution (0.05 M). Na₂SO₃ is used as a sacrificial agent. Then, the obtained suspension was sealed in a 30 mL borosilicate tube and vacuumed, and blown with Ar for about 5 minutes to completely remove air. In the dark conditions, CO₂ gas was introduced for 30 minutes to saturate the water with CO₂. Then, the borosilicate tube was placed in the center of the ultrasonic cleaner and ultrasonic machine (45 KHz, 300 W) was turned on in the dark conditions for piezoelectric catalytic reduction of CO₂. In order to detect the liquid product, 400 μL liquid inside the borosilicate tube was filtered through a filter head (0.22 μm), and 80 μL D₂O and 20 μL DMSO was added and mixed evenly and filled into a nuclear magnetic tube for 1H-NMR detection of the product. Among them, D₂O was used as the solvent, DMSO was used as the internal standard, and the ¹H-NMR (Bruker AVANCE NEO 400 MHZ) signal of the product was integrated and normalized relative to DMSO to calculate the product concentration.

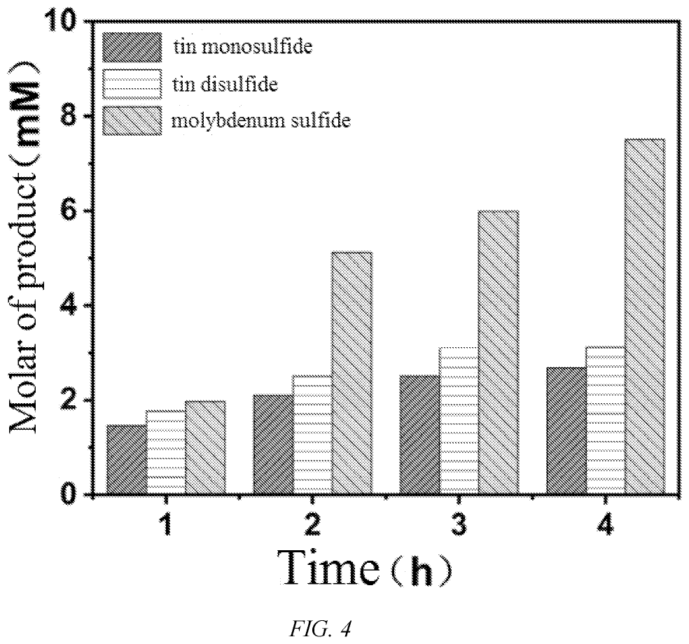

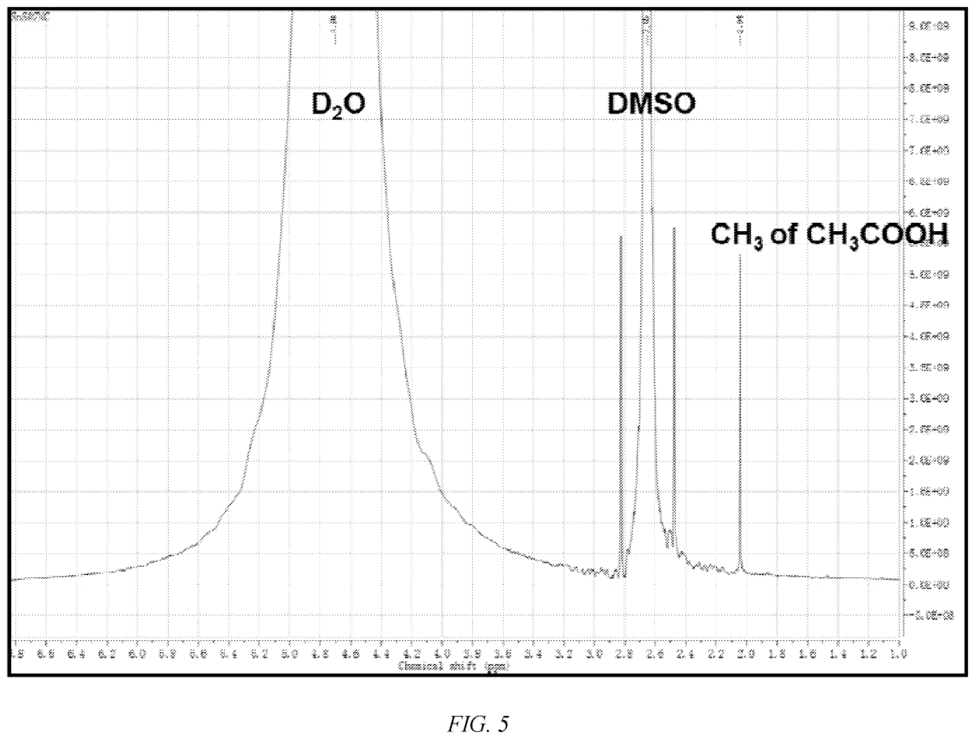

The metal sulfides prepared in Examples 1 to 5 were subjected to the above piezoelectric catalytic experiment of CO₂, and the obtained product was acetic acid. FIG. 4 shows the effect diagram of the piezoelectric catalytic reduction of CO₂ by molybdenum sulfide, tin disulfide, and tin monosulfide prepared in Examples 1, 3, and 4, and it can be seen that the amount of product of acetic acid continuously increased with the increase of catalytic time. The generating rate of acetic acid through the piezoelectric catalytic reduction of CO₂ by the molybdenum sulfide prepared in Example 1 reached 2.01 mmol g⁻¹ h⁻¹, and the nuclear magnetic resonance image of the product is shown in FIG. 5. The amounts of acetic acid obtained from the piezoelectric catalytic reduction of CO₂ by cadmium sulfide in Example 2 and zinc sulfide in Example 5 were 1.33 mmol (1 hour) and 1.36 mmol (1 hour), respectively.

With reference to the bismuth ferrite nanowire doped with manganese (10%) prepared in CN112774689A, no product of acetic acid as detected when it was subjected to the same piezoelectric catalytic reduction of CO₂. And the generating rate of acetic acid through the same piezoelectric catalytic reduction of CO₂ by the strontium titanate nanofibers doped with vanadium (0.5 mol %) previously disclosed by the inventor was 0.28 mmol g⁻¹ h⁻¹ (1 hour).

The present invention discloses a reaction system that utilizes only mechanical energy to selectively reduce CO₂ to C2+product (acetic acid) through piezoelectric catalysis. When piezoelectric nanomaterials are subjected to mechanical stimulation, active electrons are excited to participate in the reduction reaction. The built-in electric field and polarization effect caused by piezoelectric property reduce the reaction barrier and improve catalytic activity. This efficient and environmentally friendly process based on piezoelectric nanomaterials by metal sulfides provides enormous potential for utilizing environmental mechanical energy to reduce CO₂.

The above Examples are the preferred modes of execution of the present invention, but the modes of execution of the present invention are not limited by the above Examples. Any other changes, modifications, substitutions, combinations, or simplifications that do not deviate from the spirit and principles of the present invention should be equivalent substitute modes and are included in the scope of protection of the present invention.

1-10. (canceled)

11. A method for piezoelectric catalytic reduction of CO₂, comprising:
introducing carbon dioxide into a solution containing a metal sulfide piezoelectric material, and
completing the piezoelectric catalytic reduction of CO₂ under a mechanical force.

12. The method according to claim 11, further comprising:
preparing a C2+ product.

13. The method according to claim 11, wherein the metal sulfide piezoelectric material is a metal sulfide, a modified metal sulfide, or a doped metal sulfide.

14. The method according to claim 13, wherein a metal of the metal sulfide is one or more selected from the group consisting of a conventional metal, a transition metal, and a precious metal.

15. The method according to claim 13, wherein the doped metal sulfide is doped with a rare earth metal; and the modified metal sulfide is modified by an organic compound.

16. The method according to claim 13, wherein the metal sulfide is prepared from a metal salt and a sulfur source.

17. The method according to claim 16, wherein the metal sulfide is prepared from the metal salt and sulfur source by a hydrothermal or solvothermal method.

18. The method according to claim 11, wherein the mechanical force includes one or more selected from the group consisting of ultrasonic, stirring, and extrusion.

19. The method according to claim 11, wherein the metal sulfide piezoelectric material comprises molybdenum sulfide, cadmium sulfide, tin sulfide, or zinc sulfide.

20. The method according to claim 12, wherein the C2+ product is ethanol or acetic acid.
